(19) United States
(12) Patent Application Publication
(10) Pub. No.: US 2011/0202034 A1
(43) Pub. Date: Aug. 18, 2011

(54) MODULAR MEDICAL FLUID HEATING APPARATUS

(75) Inventor: James T. Lopez, Dallas, TX (US)

(73) Assignee: ESTILL MEDICAL TECHNOLOGIES, INC., Dallas, TX (US)

(21) Appl. No.: 12/891,463

(22) Filed: Sep. 27, 2010

Related U.S. Application Data
(60) Provisional application No. 61/305,377, filed on Feb. 17, 2010.

(51) Int. Cl. A61F 7/00; H05B 1/00

(52) U.S. Cl. 604/506; 219/494; 219/482

(57) ABSTRACT

A controller and a plurality of concatenated heating modules in electrical communication with the controller. The controller controls the flow of current to the plurality of heating modules in response to temperature measurements from at least one of the plurality of heating modules. In one embodiment, each of the plurality of heating modules concatenated with another heating module is connected by Luer-Lok™ connectors. In another embodiment, the modular medical fluid heating system further includes an auxiliary heating unit in electrical communication with the controller and in physical juxtaposition with the plurality of heating modules. The invention also relates to a heating module for a modular medical fluid heating system including a fluid input port; a fluid output port; and serpentine tubing through which fluid passes from the fluid input port to the fluid output port. In one embodiment, the heating module can be concatenated with other heating modules.

### RELATED APPLICATIONS

This application claims priority to and the benefit of provisional application Ser. No. 61/305,377, filed on Feb. 17, 2010, the entire contents of which are hereby incorporated by reference herein.

### FIELD OF THE INVENTION

The invention relates generally to a medical fluid dispensing apparatus and more specifically to a medical fluid heater for intravenous dispensing.

### BACKGROUND OF THE INVENTION

Patients requiring an intravenous administration of fluids, be it blood, plasma, plasma extenders or other high volume medications stand the chance of becoming hypothermic if the fluid, which is usually refrigerated or at most at room temperature, is administered without heating. This is especially critical when intravenous administration takes place in the field, away from a controlled environment such as a hospital, or when delivering fluids to children or critical care patients.

Additionally, the amount of fluid needing to be administered and the rate of administration need to be matched to the heating device. Available heating devices are typically fixed in size and do not allow for such matching.

The present invention addresses these issues.

### BRIEF DESCRIPTION OF THE DRAWINGS

The objects and features of the invention can be understood more completely by referring to the drawings described below and the accompanying descriptions.

FIG. 1 is a block diagram of an embodiment of a dispensing device constructed in accordance with an illustrative embodiment of the invention;

FIG. 2 is a schematic diagram of a general embodiment of a heating module of the dispensing device shown in FIG. 1;

FIG. 3 is a schematic diagram of the embodiment of the device shown in FIG. 1; and

FIG. 4 is a perspective view of various heating module components arranged so as to have a 3-D fluid path.

### SUMMARY OF THE INVENTION

The invention relates to medical fluid infusion warmer technology such as used for blood, IV fluid, volume medicaments and nutrients (generally referred to as medical fluids) that is easily integrated with existing medical systems and is capable of utilizing multiple power sources (including fuel cells). In addition the system has a reduced weight and size form factor that is suitable for forward combat operations. The invention includes, in one embodiment, a controller and a plurality of concatenated heating modules in electrical communication with the controller. The controller controls the flow of current to the plurality of heating modules in response to temperature measurements from at least one of the plurality of heating modules. In one embodiment, each of the plurality of heating modules concatenated with another heating module is connected by Luer-Lok™ connectors. In another embodiment the modular medical fluid heating system further includes an auxiliary heating unit in electrical communication with the controller and in close physical juxtaposition with the plurality of heating modules.

In another aspect, the invention relates to a heating module for a modular medical fluid heating system including a fluid input port; a fluid output port; and a serpentine tube through which fluid passes from the fluid input port to the fluid output port. The heating module can be concatenated with other heating modules to form a longer fluid path. In one embodiment the fluid input port and the fluid output port include Luer-LokTM connectors.

In another aspect the invention relates to a method of heating a medical fluid comprising the steps of: providing a system comprising: a controller; and a plurality of concatenated heating modules in electrical communication with the controller, each heating module comprising a tube and a heating element along the tube, and an input port and an output port; and connecting an input port of a first heating module of said plurality of heating modules to a medical fluid supply; and connecting an intravenous needle to the output port of a last heating module of said plurality of heating modules. In one embodiment the controller controls a flow of current to the plurality of heating modules in response to temperature measurements from at least one of the plurality of heating modules. In another embodiment the method further comprises the step of adding additional heating modules to the plurality of heating modules if additional heating is required.

### DESCRIPTION OF A PREFERRED EMBODIMENT

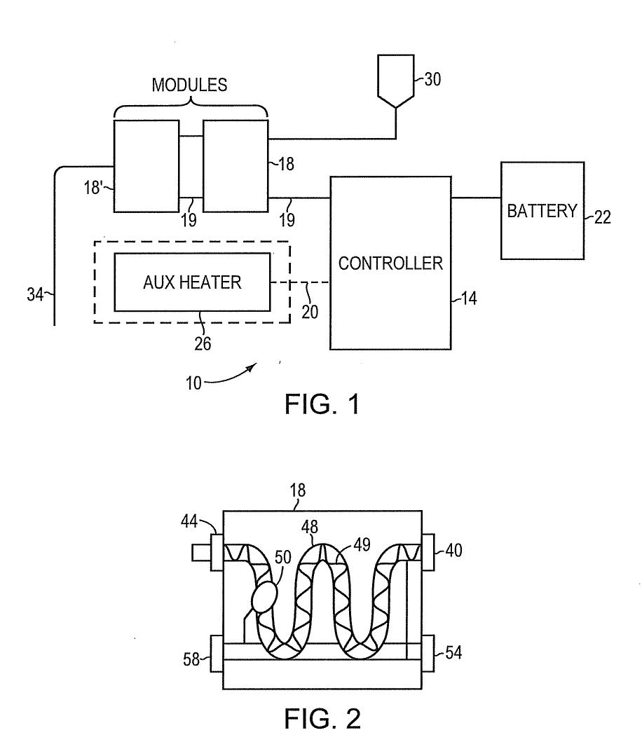

In brief overview and referring to FIG. 1, a fluid heating system 10 constructed in accordance with the invention includes a controller 14, a plurality of heating modules 18, 18′ (generally 18) and a portable power supply 22. In one embodiment, the system 10 also includes an optional auxiliary heating unit 26.

The portable power supply 22, such as a battery, is connected to controller 14. The power supply can be, without limitation, a fuel cell, a standard battery, a lithium ion battery, a solar cell, a lead acid battery suitable for supplying power to a vehicle, or the vehicle power itself. In addition the power supply source can be an AC source. The controller 14 is, in turn, connected to the heating modules 18 which are concatenated in a daisy-chain or otherwise linked or connected together. A fluid reservoir 30, such as an IV bag, is attached to the input port of the first of the series of heating modules 18 by way of a Luer-Lok™ connector. An intravenous needle assembly is attached to the output port of the last heating module 18′ of the series of modules which are in fluid communication with each other. Although this embodiment is described in terms of Luer-Lok™, connectors any removable connector can be used.

Fluid which moves from the reservoir 30 through the heating modules 18 is heated by internal electrically energized coils or resistive elements located within the module 18. Although internal electrically energized coils or resistive elements are described herein, any suitable heating element can be used. The temperature of the fluid is monitored as it passes through the series of modules 18. The temperature of the fluid is regulated by the controller 14 by controlling the amount of current passing from the power supply 22 to the heating coils of the module 18 in response to thermal detectors located along the fluid path within the heating module 18. In the embodiment shown, a heating module 18 may be concatenated or otherwise connected with other heating modules 18 to form a longer fluid path or may be used individually. These heating modules may be disposable. Fluid leaving the last heating module 18 passes through the needle set to the intravenous needle 34. Although this embodiment depicts a series of serpentine cylindrical tubes, the fluid path may be constructed as any shaped conduit or a conduit having any cross-sectional shape, including but not limited to elliptical or rectangular, provided there is a large enough surface area to volume ratio. Without the loss of generality the terms tube and conduit are used interchangeably to describe any of these conduit configurations.

In one embodiment, the system includes an optional auxiliary heating module 26. This heating module 26, in one embodiment, uses a combustible fuel source, such as methanol, to provide the thermal energy for heating the fluid. When the auxiliary heating module 26 is used, the portable power supply 22 is not used and can be disconnected from the circuit. In such an embodiment, an integral battery in the controller 14 powers the controller 14.

The auxiliary heating module 26, when in use, is positioned adjacent the heating module 18 and heat from the auxiliary heating module 26 passes through the wall of the heating module 18 and heats the fluid path of the heating module 18 by conduction. The auxiliary heating unit 26 is electrically connected 20 to the controller 14, which monitors the temperature of the fluid exiting the heating module 18 and controls the combustion in the auxiliary heating unit 26 by providing temperature data to the auxiliary heating unit 26 as a control system input.

Referring to FIG. 2, each heating module 18 includes a fluid input port 40 and a fluid output port 44. In one embodiment, the fluid ports 40, 44 are female and male Luer-Lok™ connectors, respectively. In other embodiments, the heating modules are connected by way of compression o-rings. The two ports are connected by a serpentine tube 48 through which fluid passes from the input port 40 to the output port 44. The serpentine tube 48 in one embodiment has a serpentine heating coil 49 arranged along the length of the tube. A thermal detector 50, such as a thermistor, is in, adjacent to, or along the fluid path to measure the temperature of the fluid. In one embodiment, the heating coil 49 is positioned on one side of the metallic tube 48. This allows the auxiliary heating apparatus to be attached to the other side of the tube 48 so as to heat the metallic tube 48 and not the coils 49.

The thermal detector 50 and the heating coil 49 are connected to the controller 14 through a multi-pin connector 54. A second multi-pin connector 58 provides electrical connections to and from the controller 14 for the next heating module 18 in the concatenated or series configuration.

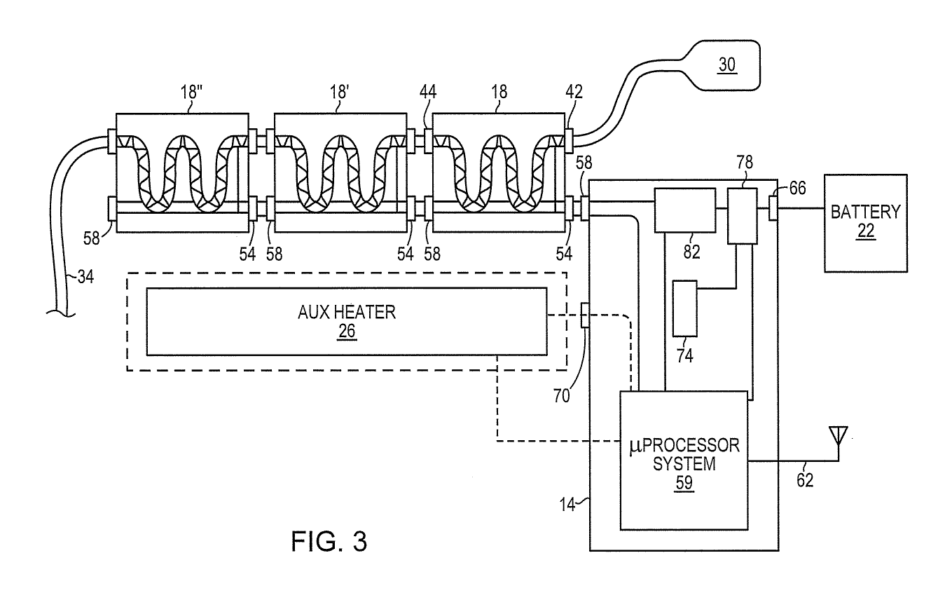

Referring to FIG. 3, in more detail, the controller 14 includes a microprocessor system 59. The microprocessor system 59 includes one or more of a ROM program memory, RAM memory, and A/D converter, a D/A converter, serial ports, parallel ports, and a wireless transceiver with antenna 62. The controller 14 also includes a connector 58 to communicate with the heating modules 18. A power connector 66 connects the power source 22 to the controller 14. A third connector 70 provides temperature information from the controller 14 to the auxiliary heating unit 26. The controller 14 also includes an internal battery 74 to supply power to the microprocessor system 59 when there is no external battery 22. Both the external power supply 22 and the internal battery 74 are connected to a power control switch 78, which disconnects the microprocessor system 59 from the internal battery 74 when the external power supply 22 is connected to the controller 14.

A power switch unit 82 which is controlled by the microprocessor system 59 connects the heating coils 49 of the heating module 18 to the power supply 22. In one embodiment, the power switch 82 is controlled by certain bits from the parallel port of the microprocessor system 59 in response to temperature data from at least one of the thermal detectors 50 (see FIG. 2) by the A/D converter of the microprocessor system 59. In one embodiment, temperature data is provided to the auxiliary heater by the D/A converter of the microprocessor system 59. It should also be noted that nothing constrains the fluid flow to three modules which is provided as exemplary only. Thus, the modules can be extended linearly.

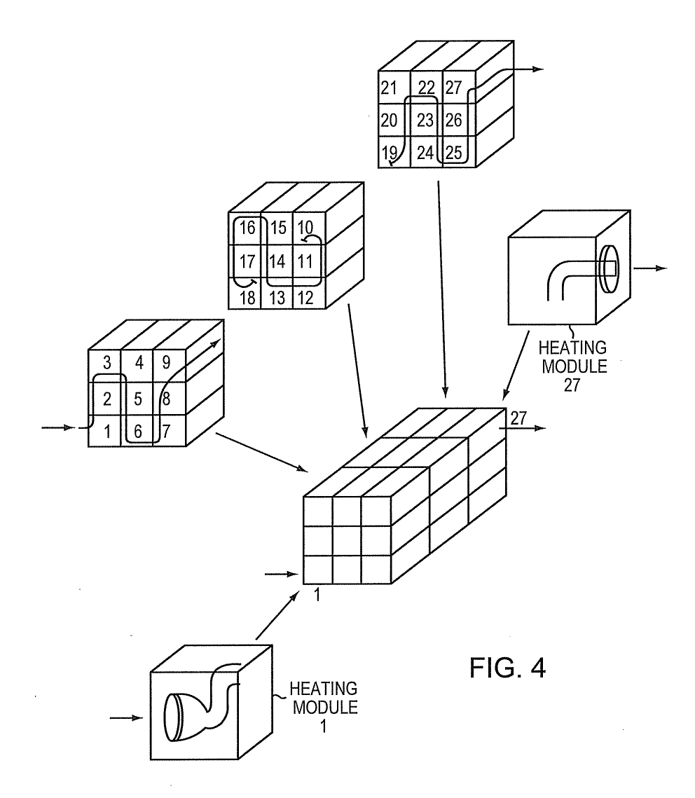

Although the system has been described in terms of a linear concatenation of heating modules, FIG. 4 discloses an equivalent system for forming adjustable fluid flow. In such a system heating modules are connected so as to have a continuous fluid path in three dimensions from the input first heating module to the twenty-seventh output heating module. The electrical connections are also continuous from the first heating module to the twenty seventh heating module. It should also be noted that nothing constrains the fluid flow to twenty seven modules which is provided as exemplary only. Thus, not only can the modules be extended linearly, but also on three dimensions.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Variations, modification, and other implementations of what is described herein will occur to those of ordinary skill in the art without departing from the spirit and scope of the invention as claimed. Accordingly, the invention is to be defined not by the preceding illustrative description, but instead by the spirit and scope of the following claims.

What is claimed is:

1. A modular medical fluid heating system comprising:
a controller; and
a plurality of concatenated heating modules in electrical communication with the controller, each heating module comprising a conduit and a heating element along the conduit;
wherein the controller controls a flow of current to the plurality of heating modules in response to temperature measurements from at least one of the plurality of heating modules.

2. The modular medical fluid heating system of claim 1 wherein each of the plurality of heating modules connected to another heating module is connected by Luer-Lok™ connectors.

3. The system of claim 1 further comprising an auxiliary heating unit capable of being in electrical communication with the controller and in close physical juxtaposition with the plurality of heating modules.

4. The system of claim 1 further comprising a portable power supply.

5. The system of claim 4 wherein the portable power supply is selected from the group of a fuel cell, a standard battery, a lithium ion battery, a solar cell , a lead acid battery, a vehicle power supply and an AC source.

6. The system of claim 1 wherein the plurality of heating modules are arranged linearly.

7. The system of claim 1 wherein the plurality of heating modules are arranged in a three dimensional arrangement.

8. A heating module for a modular medical fluid heating system comprising:
a fluid input port;
a fluid output port;
a conduit through which fluid passes from the fluid input port to the fluid output port, the conduit having a heating element; and
wherein the heating module can be concatenated using the fluid input port and fluid output port with other heating modules to form a longer fluid path.

9. The heating module of claim 8 wherein the fluid input port and the fluid output port comprise Luer-Lok™ connectors.

10. The heating module of claim 8 wherein the heating module is capable of being powered by a portable power supply.

11. The heating module of claim 10 wherein the portable power supply is selected from the group of a fuel cell, a standard battery, a lithium ion battery, a solar cell, a lead acid battery, a vehicle power supply and an AC power source.

12. The heating module of claim 8 wherein the heating module may be combined with other heating modules arranged in a linear fashion.

13. The heating module of claim 8 wherein the heating module may be combined with other heating modules arranged in a three dimensional arrangement.

14. A method of heating a medical fluid comprising the steps of:
providing a system comprising:
a controller; and
a plurality of concatenated heating modules in electrical communication with the controller, each heating module comprising a conduit and a heating element along the conduit, and an input port and an output port; and
connecting an input port of a first heating module of said plurality of heating modules to a medical fluid supply; and
connecting an intravenous needle to the output port of a last heating module of said plurality of heating modules;
wherein the controller controls a flow of current to the plurality of heating modules in response to temperature measurements from at least one of the plurality of heating modules.

15. The method of claim 14 further comprising the step of adding additional heating modules to the plurality of heating modules if additional heating is required.

16. The method of claim 14 further comprising the step of connecting the system to a portable power supply.
